(19) United States
(12) Patent Application Publication
(10) Pub. No.: US 2005/0033194 A1
(43) Pub. Date: Feb. 10, 2005

(54) Method and apparatus for sampling and collection of cervical cells

(76) Inventor: Nathan R. Fischer, Bloomfield, CT (US)

Correspondence Address: Richard C. Woodbridge, Esq.;Synnestvedt Lechner & Woodbridge, LLP, P.O. Box 592, Princeton, NJ, 08542-0592, US

(21) Appl. No.: 10/900,725

(22) Filed: Jul. 28, 2004

Related U.S. Application Data
(60) Provisional application No. 60/493,969, filed on Aug. 9, 2003.

(51) Int. Cl. A61B 10/00

(52) U.S. Cl. 600/562

(57) ABSTRACT

A method and apparatus which improves the removal of cells from a cell sampling collection device used for uterine cervical pathology screening which may be inserted into or be an integral part of the collection vial containing preservative solution. The device may be constructed of a variety of materials, such as plastic, so long as the surface is smooth to minimize cell adhesion. The distal end of the device contains an aperture or slots in the base which allows the brush or other collecting device to be inserted into the preservative liquid through the slot and the sample to be removed by rubbing the brush against the edges of the slot. The proximal end of the device may have a mechanism for anchoring or seating it in the vial so it doesn't move when the brush or other collection device is rubbed against the slot to remove cells. The device may be anchored to the vial by screwing, snapping or press fitting it against the rim or sidewalls of the vial. After the sample is taken, and the cells removed from the brush or other collecting device, the device is discarded.

### CROSS REFERENCE TO RELATED APPLICATIONS

This application claims the priority of U.S. Provisional Patent Application Ser. No. 60/493,969 filed Aug. 9, 2003 by Nathan R. Fischer and entitled “Method and Apparatus for Sampling and Collection of Cervical Cells” the entire contents and substance of which is herby incorporated in total by reference into this patent application.

### BACKGROUND OF THE INVENTION

1. Field of the Invention

This invention relates to an apparatus and method of sampling uterine cervical cells and that employs a device that fits within a sample bottle and scrapes cells from the brush or other devices used for sampling cells from the uterine cervix. (spatula, broom)

2. Description of Related Art

A Papanicolaou smear test, commonly referred to as a “Pap test,” has long been established as a highly useful diagnostic tool that allows the identification of premalignant and malignant cells at very early stages of disease. The American Council of Clinical Pathologists recommends women have an annual Pap test.

A Pap test is a clinical procedure in which a bivalve speculum is inserted into a vaginal cavity and the cervix is exposed for sampling. A sample smear of cervical cells is taken using a brush, scraper, probe or similar type of device. The collected sample, preferably taken to include cells from the squamous/columnar interface (Transformation or “T” zone) is applied to a microscope slide (a smear) and read manually or is deposited into a preservative solution to be read by an automated processing system. Two such automated liquid-based systems are manufactured by Cytyc Corporation (Boxborough, Mass.) and TriPath Imaging (Burlington, N.C.). These liquid-based systems have become the standard of care within the past 10 years. According to this prior art method, the collection device containing the sample is inserted into the vial containing a preservative solution and a brush or other sampling device is whisked around in the solution in an attempt to remove the cells from the brush and deposit them in the solution.

In spite of these advancements, close to half a million women develop cervical cancer annually and more than 50% will die. In fact, experts observe a substantial portion of cases of invasive cervical cancer in patients who are regularly screened, particularly young women. The factors that determine the success of cervical cancer screening are coverage of the population at risk for the disease, “voluntary” screening and the quality and assurance of the sample collection and the interpretation of results. The major reasons why cervical cancers are often undetected are the long intervals between tests and the high number of false negatives. These false negatives are caused by poor quality of samples and inappropriate interpretation of results. Such limitations can be overcome by improved sampling and better training of personnel.

It has been established that inadequate samples for the detection of cervical pathology results in 10-30% false negatives upon testing. Data provided by Cytyc Corporation in the ThinPrep Operators Manual indicate that 17.9% of the samples submitted to the laboratory for evaluation were limited by inadequate sampling (Endocervical Component Absent and Scant Squamous Epithelial Component). Further, 1.8% of the samples submitted were unsatisfactory for evaluation, at all, for the same reasons.

A typical sample may contain hundreds or thousands of cells and lurking in these cells may be as few as a dozen abnormal cells. Therefore, the greater the number of cells that can be collected the greater the probability that abnormal cells will be collected and will be identified either by manual visual observation or by automated reading.

The prior art includes designs which are an integral part of the vial. Such designs are not compatible with the current standard of care which requires that vials be free of internal obstructions.

The present invention relates to a new and improved method and apparatus for the removal of cells from a cervical sampling device used in the Pap test such that a greater number of cells may be removed from the sampling device and, therefore, would be available for counting and observation to detect abnormalities. By providing a sample containing a larger number of cells, the probability of “finding” abnormal cells should be increased resulting in fewer false negatives.

### SUMMARY OF THE INVENTION

The present invention comprises a new device and method for removing cells from a sampling device and depositing them into a vial containing preservative which will be sent to a laboratory for processing.

The invention may be an integral part of or an insert into existing vials such as used for the deposit and transport of samples for cervical pathology screening. The device preferably contains one or more slots or openings large enough to allow the bristles of a brush or other apparatus used for sample collection to fit in and to rub against the edges of the device thereby facilitating removal of the cells from the sample collection device. A slot or slots should run the entire diameter of the vial to allow the brush or other collection device to be moved back and forth through the preservative liquid, thereby facilitating removal of the cells. The slots may be of the same or variable width.

When contained in the vial, whether integral thereto or inserted into, the distal tip of the device should reach into the preservative liquid far enough so that the liquid covers the point of contact between the brush tip and distal rubbing bevel of the device.

If the device is employed as an insert, it should include a locking mechanism at the proximal end (screw, snap, pressure or other) which would allow it to be placed into the vial and “secured” into place at the top of the vial so that it could not be moved or inadvertently be removed while the sample collection device is being rubbed against the distal end for the purpose of sample removal into the preservative liquid. After the sample has been taken, the device is removed by releasing it (for example, “unsnapping” or “unscrewing”) it from the sample collection vial. It is then discarded.

In the center of the base of the device is the working slot which is beveled or angled such that the angle created at the point of contact between the sample collection device and the invention device tip is 90 degrees or greater thus creating a “rubbing” effect to remove cells from the sample collection device.

The material of composition of the device should be such that the surface can be rendered smooth to reduce the adhesion of cells to the device.

The invention may be more fully understood by reference to the following drawings:

### BRIEF DESCRIPTION OF THE DRAWINGS

FIG. 1A illustrates the preferred embodiment of the present invention, which consists of two stop wings and two lateral arms meeting at a common base having a bristle scraping aperture therein.

FIG. 1B illustrates the method of insertion of the invention into a standard liquid-based cytology collection vial.

FIG. 1C illustrates the invention as received in the sample collection vial and the position of the various elements of the invention relative to the vial and preservative solution in the sample collection vial.

FIG. 1D is a detailed view of the working slot and beveled edge contained in the base of the invention and showing the bristles immersed in the preservative solution as also seen in FIG. 1A.

FIG. 2 is a cross-sectional view illustrating the invention inserted into a sample collection vial and the location of the distal end relative to the level of preservative solution in the vial and as a sample collection brush is being lowered into the vial.

FIG. 3 is a cross-sectional view of the invention in a sample vial with the sample collection bristles inserted through the working slot and submerged into the preservative solution.

FIG. 4 is a cross-sectional view of the invention in a sample collection vial and showing the sample collection brush being removed from the slot or aperture with cellular debris being left behind in the preservative solution.

FIG. 5 and FIG. 6 are detailed views of the sample collection brush being scraped against the beveled edge of the working slot thus optimizing removal of the cellular material from the brush.

FIGS. 7-9 illustrate alternative embodiments of the aperture or slot showing other possible configurations of the base and working slot.

FIGS. 10-11 illustrate alternative embodiments of the invention, which would allow the invention to be self-contained in a vial and a cap to be screwed on with the device in the sample collection vial.

FIG. 12 illustrates another alternative embodiment of the invention in which the stop wings are vertical and the invention is secured to the vial by means of a clamp over the threads of the vial.

FIG. 12A illustrates the alternative embodiment of FIG. 12 installed in a sample collection vial.

### DETAILED DESCRIPTION OF THE PREFERRED EMBODIMENT

During the course of this disclosure, like elements will be identified by like numbers according to the different views that illustrate the invention.

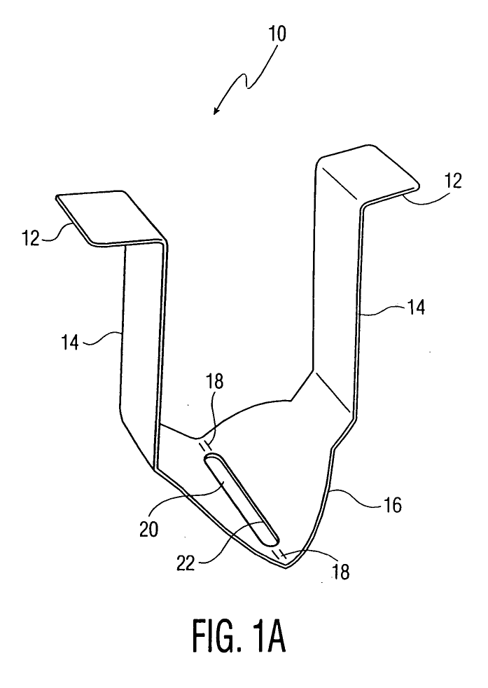

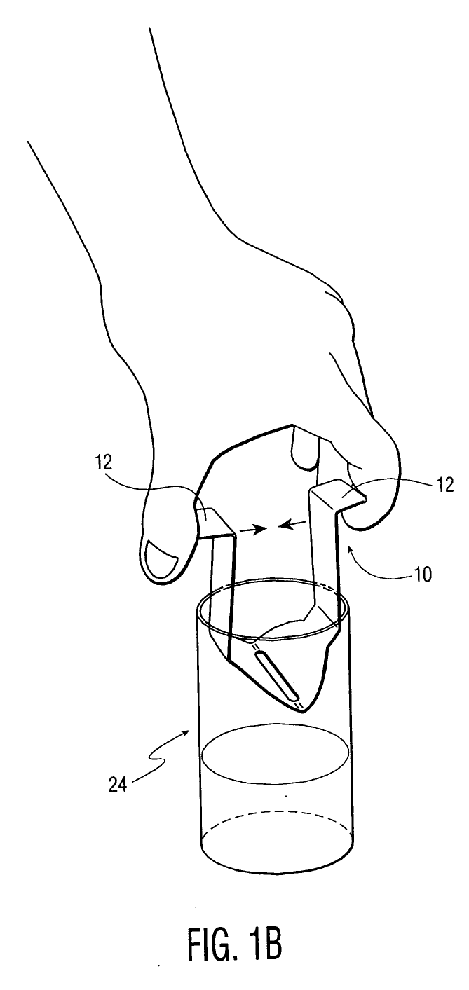

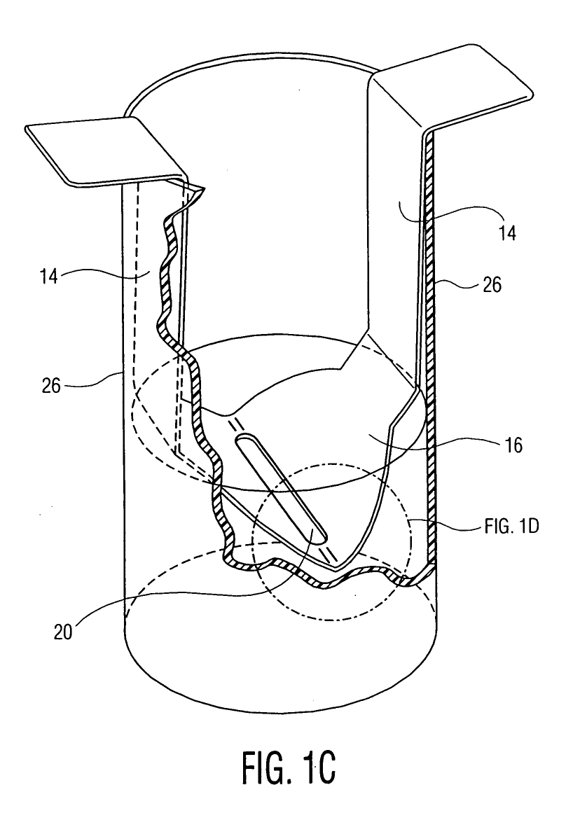

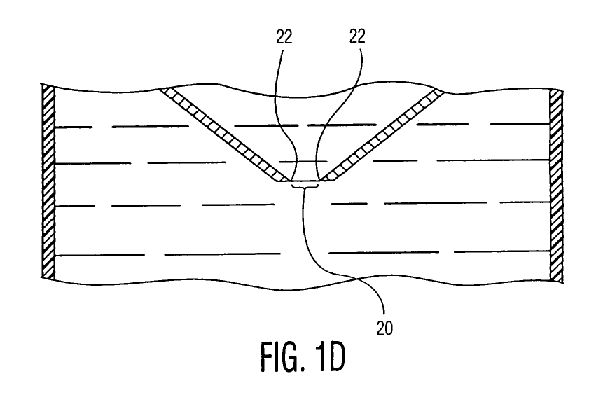

Referring first to FIG. 1A, the preferred embodiment of the device comprises an insert 10 for use with a liquid sample collection vial 24 for cervical cytology testing. Vial 24 includes a bottom, sidewalls and a rim. The invention 10 includes stop wings 12, arms 14 and a base 16 united by base connectors 18 on each side. In the middle and bottom of the base is a working slot 20 with a beveled edge 22. The width of each arm 14 is approximately 23 mm and the base connectors 18 are 3 mm in width. The base 16 is angled at approximately 90 degrees. The diameter of the base 16 is approximately 30 mm and the circumference is approximately 94.2 mm. The working slot 20, located in the base 16, is 22-25 mm in length; the width is approximately 2.0-2.1 mm and is rounded at the ends. The invention may be constructed of 302 bright finish stainless steel with a thickness of 0.5-1.0 mm.

As shown in FIG. 1B, the invention 10 is insertable into a sample collection vial 24 by squeezing the stop wings 12 together toward each other with thumb and forefinger and then pushing the invention 10 into the vial 24.

In FIG. 1C, the arms 14 are shown nearly parallel to the side wall 26 of the vial 24. The base 16 does not touch the sides 26 of the vial, but occupies most of the circumference of the vial. The length of the arms 14 can be variable based on the depth of the solution in the vial.

In FIG. 1D, the working slot 20 is shown in a detailed view to magnify the beveled edge 22.

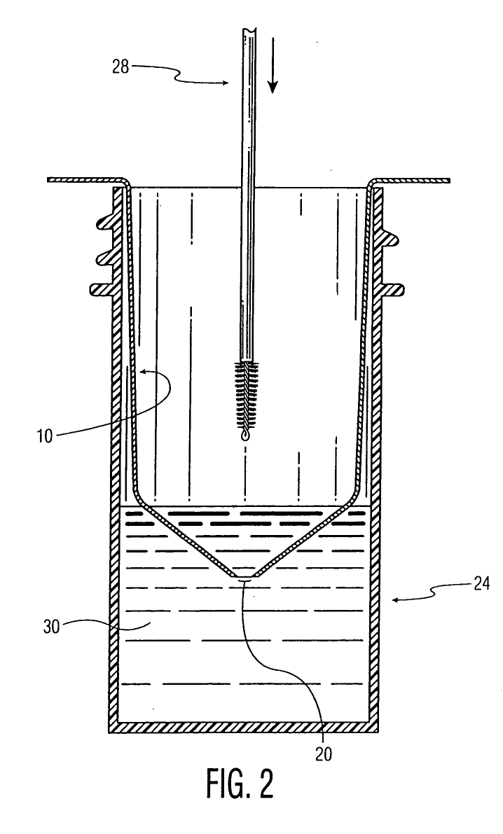

The invention 10 is shown in the sample collection vial 24 in FIG. 2. A sample collection device 28 is about to be inserted into the working slot 20 which is 2-3 mm beneath the surface of the preservative liquid 30.

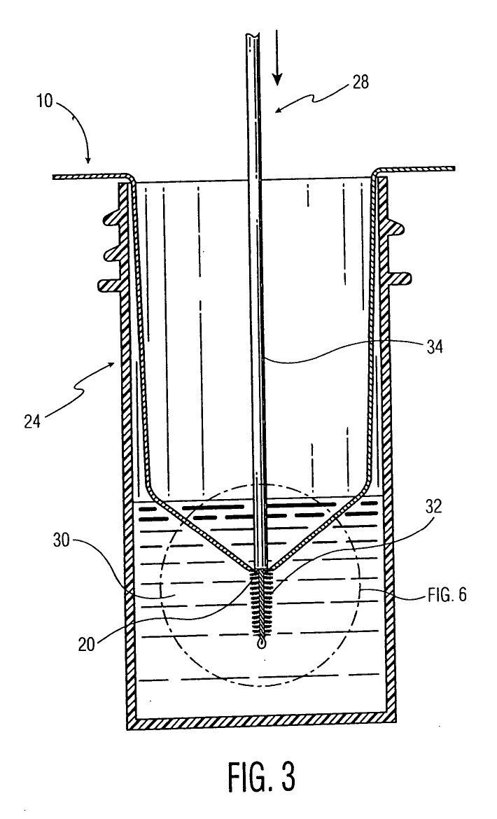

As seen in FIG. 3, the width of the working slot 20 is such that it allows the brush bristles 32 of the sample collection device 28 to reach into the preservative liquid 30 but not extend past the handle 34 of the sample collection device 28. When the invention 10 is inside the vial 24, with the edges engaged against the side wall 26 of the vial 24, the invention 10 is held in place by a lateral pressure of 6-12 ounces. This lateral pressure holds the invention 10 in place when the sample collection device 28 is rubbed against the working slot 20.

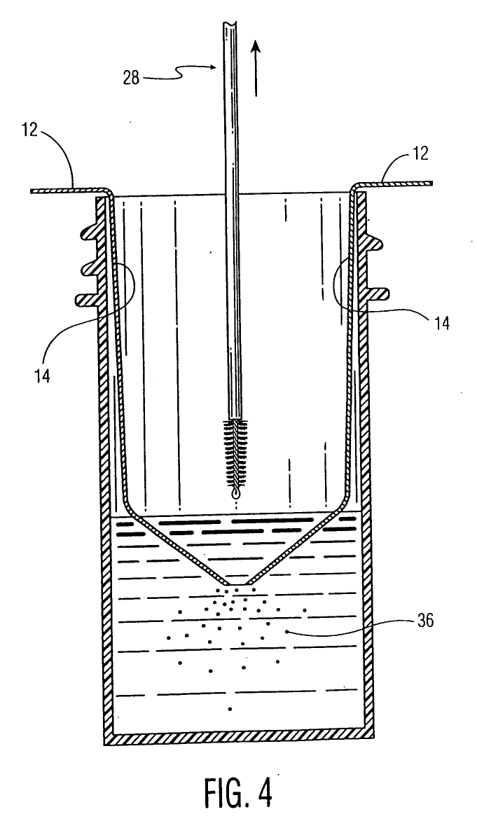

FIG. 4 illustrates the stop wings 12 at a 90 degree angle to the arms 14 and abutting the rim of the vial 24. The stop wings 12 are intended to stop the invention from going deeper into the vial thereby allowing for optimal collection of cells from the collection device. FIG. 4 also illustrates the sample collection device 28 being removed from the vial 24 with the cellular material 36 being left behind in the preservative solution.

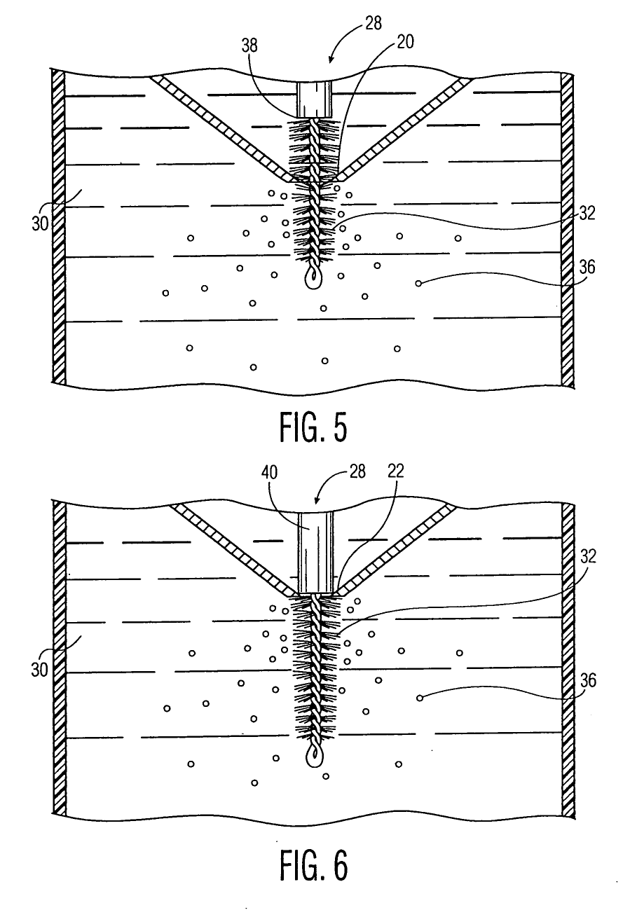

Greater detail can be seen in FIGS. 5 and 6. In FIG. 5, the bristles 32 of the sample collection device 28 are being inserted through the working slot 20 of the invention and, as it is withdrawn, the bristles 32 rub against the beveled edge 22 thus removing the cellular material 36 into the preservative solution 30. FIG. 6 shows how bristles 32 of the sample collection device 28 cannot be inserted through the working slot 20 past the junction 38 of the handle 40. The diameter of the handle region 40 is preferably larger than the width or diameter of working slot 20 so as to limit the travel of the bristles into the vial 24.

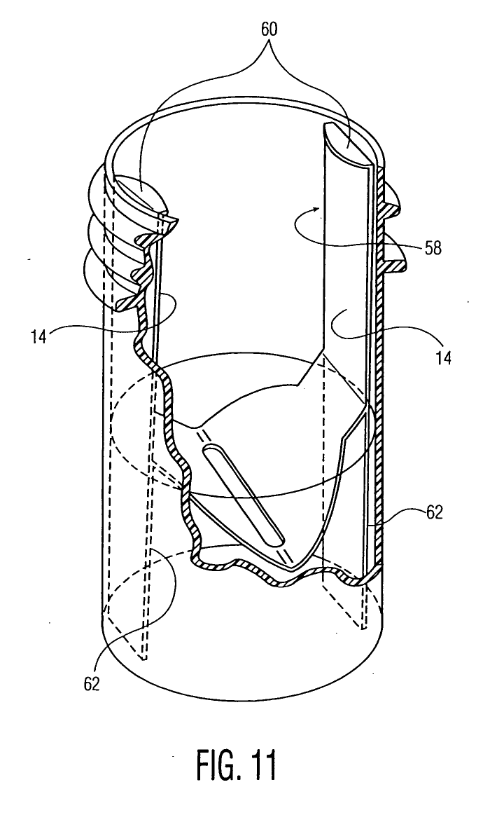

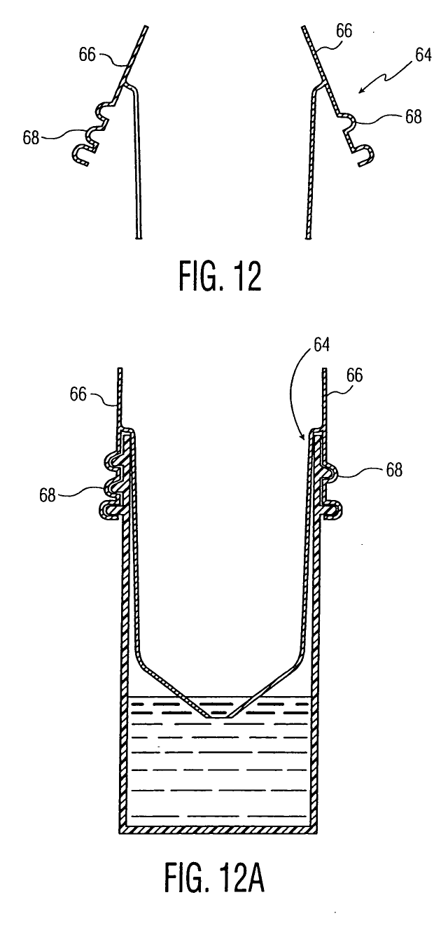

After use, the stop wings 12 are squeezed toward the center, as is shown in FIG. 1B or 12, thus releasing lateral pressure from the edges of the vial 24 and allowing the device 10 to be pulled out, removed and discarded.

The invention 10 is used according to the following steps:

The user would first remove the cap from the vial 24, as is normally done. According to the preferred embodiment of the invention, once the cap is removed, the user would first squeeze the stop wings 12 together toward the center using thumb and forefinger and insert the invention 10 into the vial 24 until the stop wings touch the top of the vial, FIG. 1B.

Second, the user would release the stop wings 12 allowing the arms to press against the sidewall 26 of the vial 24 thus holding the invention 10 in place with the pressure against the sidewall 26.

Third, the user would obtain the sample from the patient in a normal fashion using whatever sample collection device is preferred (brush, spatula, etc).

Fourth, the user would place the sample collection device 32 into the vial 24 containing the invention 10, as shown in FIG. 2. The structure of the invention is such that the bristles 32 of the sample collection device 28 will be directed into the working slot 20 by the 90 degree angle of the base 26. The user would move the bristles 32 back and forth and up and down in the preservative liquid along the length of the working slot 20. This action allows the bristles 32 to scrape against the beveled edge 22 of the working slot 20 thus facilitating removal of the cells from the collection device as seen in FIGS. 4-6.

Finally, the user would squeeze the stop wings 12 together, as in FIG. 1B, which would release the arms 14 from the sides of the vial 24 thus allowing the insert 10 to be removed and discarded.

### Alternative Embodiments of the Invention

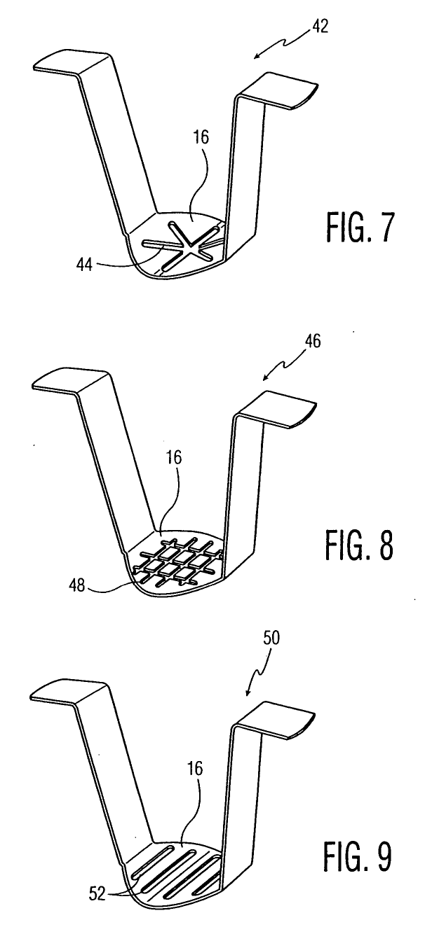

Alternative embodiments of the slot or aperture 20 are shown in FIGS. 7-9.

FIG. 7 illustrates an alternative embodiment of the invention 42 with a base 16 that has a working slot 44 in the shape of a 6-point star.

FIG. 8 illustrates the invention 46 with a grid pattern in the base 16 for the working slot 48.

FIG. 9 illustrates an alternative embodiment of the invention 50 with several working slots 52 in the base 16 which run parallel to each other.

Each of the foregoing alternative embodiments contain the key elements of the invention 10 such as a working slot, beveled edges and stop wings.

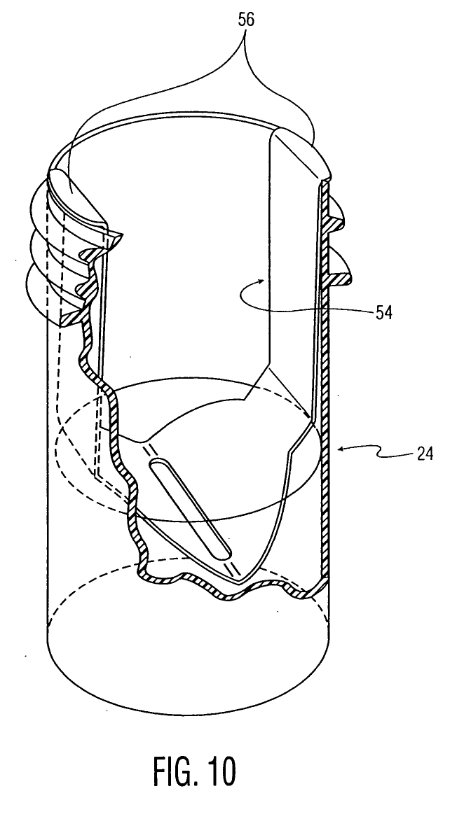

FIGS. 10 and 11 illustrate alternative embodiments of the invention which would allow the invention to be self-contained in the vial 24 and would preclude the need for the user to insert it prior to use. However, it would still be possible to remove the alternative embodiment and discard it after use so as to not interfere with the subsequent analysis process.

FIG. 10 illustrates the invention 54 with modified stop wings 56 which do not protrude past the outside diameter of the rim of the sample collection vial 24. This embodiment allows the invention to be loaded into the vial 24 and sealed with the cap prior to shipping to the user. This also eliminates the need for the user to have to insert the invention prior to use. However, there is enough of an edge over the top of the vial for the user to squeeze the stop wings together for removal after use.

FIG. 11 illustrates another embodiment of the invention 58 which allows it to be self-contained in the vial 24 prior to use. In this embodiment, the stop wings 60 protrude inward, toward the center of the vial, approximately 1-2 mm. This provides a point for grasping the invention 58 for removal after use. Since the stop wings 60 do not rest on the top rim of the vial 24 for support, the appropriate height of the invention, in the preservative solution, is achieved by extending the arms 14 all the way to the bottom of the vial by means of arm extensions 62.

FIGS. 12 and 12A illustrates an alternative embodiment of the invention 64 showing the stop wings 66 in a vertical position which, when squeezed toward each other, allows the stop wing extensions 68 to be opened to permit them to be engaged onto the external threads at the top of the vial 24, thus securing the invention to the vial to the appropriate depth into the preservative liquid.

Other methods of attaching the invention to the vial, such as screwing, snapping or otherwise engaging it onto the vial so it is not easily removed during use may also be employed.

The invention is preferably manufactured from a suitable plastic such as polypropylene. The strength of the plastic should be sufficient so that it stays in place in the sample collection vial during use, but can be easily removed and discarded.

While the invention has been described with reference to a preferred embodiment thereof, it will be appreciated by those of ordinary skill in the art that various changes can be made to the structure and steps that comprise the invention without departing from the spirit and scope of the invention as a whole.

1. A sample collection apparatus for collecting cervical cells from the bristles of a collection brush and suitable for insertion into a cervical cell sample collection vial having a bottom, sidewalls and a rim, said apparatus comprising:
a base;
an aperture in said base;
a first arm having a first and a second end and wherein said first end of said first arm is attached to said base; and,
a second arm also having a first and a second end and wherein said first end of said second arm is also attached to said base,
wherein said apparatus is insertable into said vial and cervical cells are removed from said bristles by pushing said bristles into said aperture.

2. The apparatus of claim 1 further comprising:
depth control means connected to said first and second arms for limiting the distance from the aperture to the bottom of said vial.

3. The apparatus of claim 2 wherein said bristles of said cervical cell collection brush are attached to a handle and wherein the width of said aperture is wider than the diameter of said bristles but narrower than said handle so that insertion of said bristles into said aperture prevents the bristles from being inserted substantially beyond said handle and further wherein said depth control means prevents said bristles from contacting said bottom of said vial.

4. The apparatus of claim 3 wherein said depth control means comprises:
a first wing attached to the second end of said first arm; and,
a second wing attached to the second end of said second arm,
wherein said first and second wings engage the rim of said vial and limit the depth of travel of said apparatus into said vial.

5. The apparatus of claim 4 wherein said first and second wings face away from each other.

6. The apparatus of claim 5 wherein said arms are resilient and are bent inward towards each other as said apparatus is inserted into said vial thereby helping to insure that said apparatus stays in said vial during use.

7. The apparatus of claim 3 wherein said depth control means comprises:
a first arm extension attached to said first arm and extending beyond said base; and,
a second arm extension attached to said second arm and also extending beyond said base,
wherein said first and second arm extensions contact said bottom of said vial when said apparatus is inserted in said vial and thereby limit the distance between said aperture and said bottom of said vial.

8. The apparatus of claim 3 wherein said vial has external threads thereon and wherein said depth control device comprises:
a first stop wing having thread engaging means thereon and attached to said second end of said first arm; and,
a second stop wing also having thread engaging means thereon and attached to said second end of said second arm,
wherein squeezing said first and second stop wings towards each other separates said thread engaging means and further wherein releasing said first and second stop wings thereafter permits said thread engaging means to engage the external threads on said vial thus securing said apparatus to said vial and limiting the distance between said aperture and the bottom of said vial.

9. The apparatus of claim 3 wherein said aperture comprises a slot.

10. The apparatus of claim 3 wherein said aperture comprises a plurality of slots.

11. The apparatus of claim 3 wherein said aperture comprised a plurality of slots that intersect in a star shaped fashion.

12. The apparatus of claim 3 wherein said aperture comprised a plurality of slots that cross each other in a grid-like fashion.

13. The apparatus of claim 3 wherein said aperture has a peripheral edge and wherein said peripheral edge is beveled to enhance the harvesting of cells from said bristles.

14. A method of collecting cervical cells from the bristles of a cervical cell brush attached to a handle region and collecting said cervical cells in a vial having a bottom, sidewalls and a rim, said method comprising the steps of:
a. inserting a cervical cell scraping means into said vial, said cervical cell scraping means having an aperture therein for receiving said bristles;
b. limiting the depth of travel of said bristles of said cervical cell scraping means into said vial,
wherein said bristles can not contact said bottom of said vial.

15. The method of claim 14 further comprising the step of:
c. limiting the depth of travel of said bristles into said aperture,
wherein said travel of said bristles into said aperture does not travel substantially beyond said bristles and into said handle region.

16. The method of claim 15 wherein said inserting step a. further includes the step of:
d. squeezing said cervical cell scraping means as it is being inserted into said vial so that when said squeezing pressure is released said cervical cell scraping means attempts to return to its natural shape and impinges upon the sidewalls and holds it in place.

17. A sample collection apparatus for collecting cervical cells from a sample collection device and suitable for insertion into a cervical cell sample collection vial having a bottom, sidewalls and a rim, said apparatus comprising:
a base;
an aperture in said base;
a first arm having a first and a second end and wherein said first end of said first arm is attached to said base; and,
a second arm also having a first and a second end and wherein said first end of said second arm is also attached to said base,
wherein said apparatus is insertable into said vial and cervical cells are removed from said sample collection device by pushing said sample collection device into said aperture.

18. The apparatus of claim 17 wherein said sample collection device comprises a brush having bristles.
